(19) United States
(12) Patent Application Publication
(10) Pub. No.: US 2016/0272884 A1
(43) Pub. Date: Sep. 22, 2016

(54) Compensation Film and Organic Dot for Compensation Film

(71) Applicant: TORAY CHEMICAL KOREA INC., Gyeongsangbuk-do (KR)

(72) Inventors: Ji Hwan Kim, Gyeonggi-do (KR); Hyo Seok Kim, Gyeonggi-do (KR)

(21) Appl. No.: 15/033,476

(22) PCT Filed: Apr. 18, 2014
(86) PCT No.: PCT/KR2014/003396

§ 371 Date: Apr. 29, 2016

(30) Foreign Application Priority Data
Nov. 1, 2013 (KR) 10-2013-0132280
Dec. 13, 2013 (KR) 10-2013-0155776

(51) Int. Cl. C09K 11/06; C07D 471/06; C07F 5/02; C09K 11/02

(52) U.S. Cl. CPC C09K 11/06; C09K 11/02; C07D 471/06; C07F 5/022; C09K 2211/1048; C09K 2211/1055

(57) ABSTRACT

The present invention relates to a novel organic dot for a compensation film and a compensation film using the same, and an organic dot of the present invention relates to a new material which not only can replace existing quantum dots (QDs), but also can improve color reproduction power regarding R (red) and G (green) and can enhance optical properties of materials such as LCD efficiency, color reproducibility and the like.

### TECHNICAL FIELD

The present invention relates to a compensation film including organic dots and organic dots for a compensation film having a specific PL wavelength.

### BACKGROUND ART

Quantum dots are a nano-size semiconductor material and a material exhibiting quantum confinement effect. The quantum dots generate stronger light in a narrow wavelength range than a common phosphor.

The light emission of the quantum dots is attained during the transition of electrons with an excited state from a conduction band to a valence band, and the quantum dots exhibit different wavelengths according to the particle size thereof even though for the same material. As the size of the quantum dots decreases, light having short wavelength may be emitted, and the light with a desired wavelength range may be obtained by controlling the size of the quantum dots.

Since the quantum dots may emit light even though selecting any excitation wavelength optionally, in the case that various kinds of quantum dots are present, lights with various colors may be observed at a time via excitation by one wavelength. Quantum dots may emit lights with different colors according to the particle size thereof even though prepared using the same material. Due to the above-described properties, the quantum dots receive much attention as a next generation light emitting diode (LED) with high luminance, a bio sensor, a laser, a nano material for a solar cell, etc.

Recently, a preparation method commonly used for the preparation of the quantum dots (Korean Laid-open Patent Publication No. 2011-0091361, publication date: Aug. 11, 2011) is a nonhydrolytic synthesis. According to the method, an organometallic compound at room temperature is rapidly injected as a precursor to a solvent with a high temperature, nuclearization using a thermal decomposition reaction is performed, and heat is applied to grow the nuclear and to produce quantum dots. The quantum dots mainly synthesized by the above method include cadmium (Cd) such as cadmium selenium (CdSe) and cadmium tellurium (CdTe). However, in consideration of the present trend pursuing green industries with high awareness on environmental issues, the use of cadmium (Cd) which is one of typical environmental contaminating materials contaminating water and soil is required to be minimized.

Therefore, as an alternative to the conventional CdSe quantum dots or CdTe quantum dots, the preparation methods of quantum dots using a semiconductor material not including cadmium are considered, and one of them uses indium sulfide (In₂S₃) quantum dots.

In particular, indium sulfide (InS₂) has a bulk band gap of 2.1 eV, and InS₂ quantum dots may emit in a visible region and may be used for the manufacture of a light emitting diode with high luminance. However, in general, since the synthesis of a material using elements in group 13 and 16 is difficult, the mass production of the indium sulfide quantum dots is difficult, and the securing of the uniformity of a particle size or a quantum yield (QY) is inferior to that of conventional CdSe.

Accordingly, the requirement of the developments of novel quantum dots not using cadmium is increasing.

Each of OLED and LCD has merits and demerits, and OLED has excellent color reproducibility of red (R), green (G) and blue (B), however has inferior resolution, and the reproduction of high resolution is deteriorated when compared to LCD. On the contrary, LCD capable of reproducing high resolution has defects of having worse RGB color reproducibility than OLED. Therefore, the requirement of technique for increasing the resolution of the OLED and/or increasing the reproducibility, the luminance and the luminous efficacy of the LCD is increasing.

### DISCLOSURE OF THE INVENTION

### Technical Problem

Accordingly, the inventors of the present invention tried to find a novel material which may replace the quantum dots which are the conventional inorganic material, and developed novel organic dots having a unimolecular shape using an organic material. That is, the present invention provides organic dots which have a specific chemical formula and a specific photoluminescence (PL) wavelength, and a compensation film using the same.

### Technical Solution

To solve the above tasks, there is provided in the present invention, a compensation film including organic dots having a unimolecular shape.

According to a preferred embodiment of the present invention, the compensation film has a photoluminescence (PL) wavelength of 500-680 nm.

According to a preferred embodiment of the present invention, the organic dots used in the compensation film of the present invention may include a compound represented by the following Formula 1 and/or a compound represented by the following Formula 2.

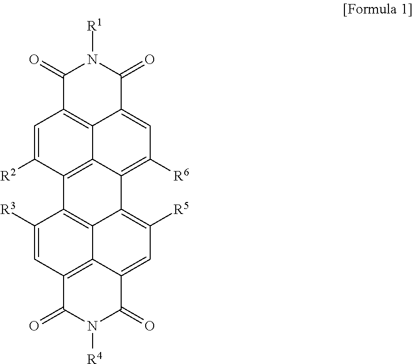

In Formula 1, R¹ and R⁴ are each independently hydrogen, linear alkyl of C1-C5, branched alkyl of C3-C5, cycloalkyl of C5-C6,

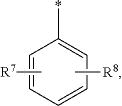

or —CN, R², R³, R⁵ and R⁶ are each independently hydrogen, alkoxy of C1-C5, cyclicalkoxy of C5-C10,

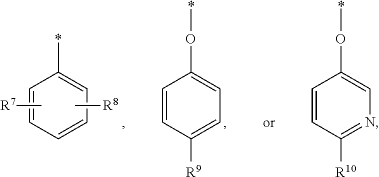

R⁷ and R⁸ are each independently hydrogen, linear alkyl of C1-C5, or branched alkyl of C3-C5, R⁹ and R¹⁰ are each independently hydrogen, —SO₃H, —COOH, —CH₂COOH, —CH₂CH₂COOH, —CH₂CH₂CH₂COOH, —NR¹¹R¹², —CH₂NR¹¹R¹², or —CH₂CH₂NR¹¹R¹², and R¹¹ and R¹² are each independently hydrogen, or linear alkyl of C1-C3,

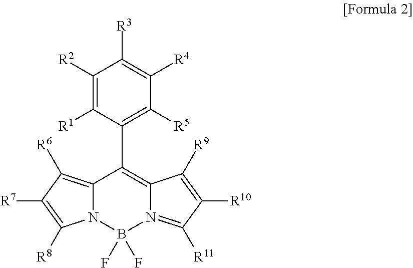

In Formula 2, R¹ to R⁵ are each independently hydrogen, alkyl of C1-C5, halogen, or —CN, R⁶ to R¹¹ are each independently hydrogen, alkyl of C1-C5, olefin of C2-C5, cycloalkyl of C5-C6, styrene, phenyl, benzyl, or —CN.

According to another preferred embodiment of the present invention, the compound represented by Formula 1 and the compound represented by Formula 2 may be included in an amount ratio of 1:0.05-20 by weight in the compensation film.

According to another preferred embodiment of the present invention, in the compound represented by Formula 1, which is one of the components of the compensation film, R¹ and R⁴ may be each independently alkyl of C1-C5, or

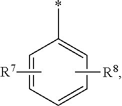

R⁷ and R⁸ may be alkyl of C2-C4, or branched alkyl of C3-C4, R², R³, R⁴ and R⁶ may be each independently cyclicalkoxy of C5-C10,

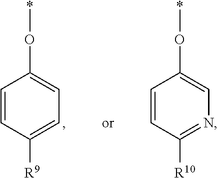

R⁹ and R¹⁰ may be each independently hydrogen, —SO₃H, —COOH, —CH₂COOH, or —CH₂NR¹¹R¹², and R¹¹ and R¹² may be each independently hydrogen or linear alkyl of C1.

According to another preferred embodiment of the present invention, in the compound represented by Formula 2, which is one of the components of the compensation film, R¹ to R⁵ may be each independently hydrogen, or alkyl of C1-C2, R⁷ and R¹⁰ may be hydrogen, R⁶, R⁸, R⁹ and R¹¹ may be each independently alkyl of C1-C2, cycloalkyl of C5-C6, styrene, phenyl, benzyl, or —CN.

According to another preferred embodiment of the present invention, the compensation film of the present invention may have an x coordinate range of 0.20-0.50, and a y coordinate range of 0.15-0.40 on the basis of an NTSC color coordinate under a blue light source.

According to another preferred embodiment of the present invention, the compensation film may further include at least one of quantum dots, polymer dots, or a dye as well as the organic dots.

According to another preferred embodiment of the present invention, an average thickness of the compensation film of the present invention may be 0.1-200 μm.

Another aspect of the present invention relates to organic dots including the compound represented by Formula 1 and/or the compound represented by Formula 2.

According to another preferred embodiment of the present invention, the compound represented by Formula 1 may have a photoluminescence (PL) wavelength of 580-680 nm, and the compound represented by Formula 2 may have a photoluminescence (PL) wavelength of 500-680 nm.

Another aspect of the present invention relates to a compensation film composition including 0.05-7 parts by weight of a luminescent material including the organic dots represented by Formula 1 and/or Formula 2, and 30-1,700 parts by weight of beads relative to 100 parts by weight of a binder.

According to a preferred embodiment of the present invention, the luminescent material may include the compound represented by Formula 1 and the compound represented by Formula 2 in a weight ratio of 1:0.05-20 in the compensation film composition of the present invention.

According to a preferred embodiment of the present invention, the binder may include at least one selected from an aliphatic urethane acrylate resin, an epoxy acrylate resin, a melamine acrylate resin, or a polyester acrylate resin in the compensation film composition of the present invention.

According to a preferred embodiment of the present invention, the beads may have an average particle diameter of 0.5-30 μm in the compensation film composition, and the beads may include at least one selected from silica, zirconia, titanium dioxide, polystyrene, polypropylene, polyethylene, polyurethane, or polymethyl(meth)acrylate.

According to another preferred embodiment of the present invention, the compensation film composition may further include at least one of quantum dots, polymer dots, or a dye as well as the organic dots.

Another aspect of the present invention relates to the use of the organic dots and/or the compensation film, and relates to a light emitting diode (LED) display, a light emitting diode (LED) illumination apparatus, and/or a liquid crystal display (LCD), including the organic dots and/or the compensation film of the present invention.

### Advantageous Effects

The present invention relates to a luminescent material without using an inorganic material such as cadmium, and not inducing environmental issues. The organic dots of the present invention has a PL wavelength of 500-680 nm and a decreasing half width (color reproducibility) of a red system and/or a green system and increasing quantum efficiency (luminous efficacy), and may replace the conventional quantum dots of an inorganic material. The organic dots of the present invention may be used per se or as an application type such as a compensation film to be used in various fields including a bio sensor, an illumination apparatus, a display, etc.

### BRIEF DESCRIPTION OF THE DRAWINGS

FIG. 1 is a graph obtained by measuring the PL wavelength of organic dots prepared in Example 1.

FIG. 2 is a graph obtained by measuring the PL wavelength of organic dots prepared in Example 2.

FIG. 3 is an NTSC color coordinate graph.

FIG. 4 is an SEM photograph of silica beads used in Preparation Example 1.

FIG. 5 a schematic diagram of an embodiment of a compensation film manufacturing in Preparation Example 1.

FIG. 6 is a schematic diagram of an embodiment of a luminance enhancing film using the compensation film manufactured in Preparation Example 1.

### BEST MODE FOR CARRYING OUT THE INVENTION

The term “film” used in the present invention has wide meaning including a sheet shape as well as a film shape commonly used in the art.

The terms “C1”, “C2”, etc. used in the present invention mean the number of carbon atoms, and for example, “alkyl of C1-C5” means “alkyl having 1-5 carbon atoms”.

In a formula represented by

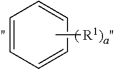

in the present invention, in the case that “R¹ is independently hydrogen, methyl, or ethyl, and a is 1-3” is written for a substituent, and in the case that a is 3, a plurality of R¹s (that is, three R¹ substituents) are present, and the plurality of R¹s may be the same or different, where each of R¹s may be hydrogen, methyl or ethyl, or each of R¹s may be different from each other, and one of R¹ may be hydrogen, another one may be methyl, and further another one may be ethyl. Here, the above explanation is an example for interpreting the substituents represented in the present invention, and analogous substituents of different shape should be interpreted by the same method.

The terms “mono dispersive” used in the present invention means that particles have the same particle diameter and the coefficient of variation (CV) of a diameter of 9% or less in the present invention. In addition, “poly dispersive” means that particles having different particle diameters are mixed, and the CV of a diameter is 20% or more in the present invention.

Hereinafter the present invention will be explained in detail.

The present invention relates to a compensation film including organic dots having a unimolecular shape, and the compensation film of the present invention may have a photoluminescence (PL) wavelength of 500-680 nm.

The organic dots used in the present invention will be explained.

The present invention relates to novel organic dots, and the organic dots of the present invention include a compound represented by the following Formula 1 and/or a compound represented by the following Formula 2.

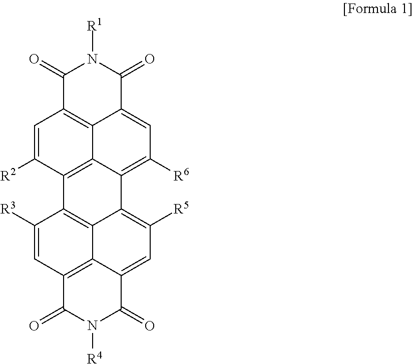

In Formula 1, R¹ and R⁴ are each independently hydrogen, linear alkyl of C1-C5, branched alkyl of C3-C5, cycloalkyl of C5-C6,

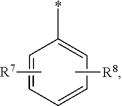

or —CN, preferably, alkyl of C1-C5, or

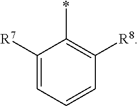

R⁷ and R⁸ are each independently hydrogen, linear alkyl of C1-C5, or branched alkyl of C3-C4, preferably, alkyl of C2-C4, or branched alkyl of C3-C4, and more preferably, branched alkyl of C3-C4.

In addition, R², R³, R⁴ and R⁶ in Formula 1 are each independently hydrogen, alkoxy of C1-C5, cyclicalkoxy of C5-C10,

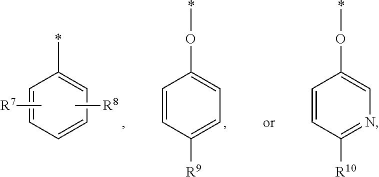

preferably, cyclicalkoxy of C5-C10,

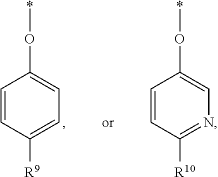

and more preferably, R², R³, R⁴ and R⁶ are each independently

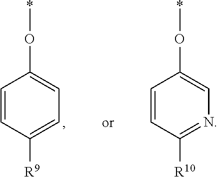

In addition, R⁷ and R⁸ are each independently hydrogen, linear alkyl of C1-C5, or branched alkyl of C3-C5, preferably, alkyl of C2-C4, or branched alkyl of C3-C4, and more preferably, branched alkyl of C3-C4. In addition, R⁹ and R¹⁰ are each independently hydrogen, —SO₂H, —COOH, —CH₂COOH, —CH₂CH₂COOH, —CH₂CH₂CH₂COOH, —NR¹¹R¹², —CH₂NR¹¹R¹², or —CH₂CH₂NR¹¹R¹², and preferably, hydrogen, —SO₃H, —COOH, —CH₂COOH, or —CH₂NR¹¹R¹². R¹¹ and R¹² are each independently hydrogen or linear alkyl of C1-C3, and preferably, R¹¹ and R¹² are each independently hydrogen or linear alkyl of C1.

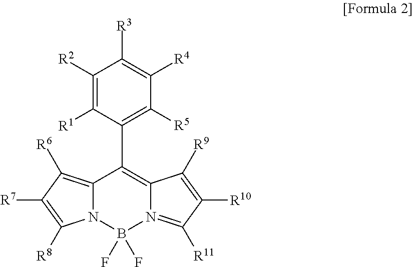

In Formula 2, R¹ to R⁵ may be each independently hydrogen, alkyl of C1-C5, one kind of halogen among —Cl, —F, —Br, or —I, or —CN, preferably, R¹ to R⁵ may be each independently hydrogen, alkyl of C1-C2, —F, or —CN, and more preferably, R² and/or R⁴ may be hydrogen and/or —CN, R¹, R³ and R⁵ may be each independently alkyl of C1-C2, —F, and/or —CN.

In addition, in Formula 2, R⁶ to R¹¹ may be each independently hydrogen, alkyl of C1-C5, olefin of C2-C5, cycloalkyl of C5-C6, styrene, phenyl, benzyl, or —CN, preferably, R⁷ to R¹⁰ may be hydrogen, R⁶, R⁸, R⁹ and R¹¹ may be each independently alkyl of C1-C2, cycloalkyl of C5-C6, styrene, phenyl, benzyl, or —CN, and more preferably, R⁷ and R¹⁰ may be hydrogen, R⁶, R⁸, R⁹ and R¹¹ may be the same and may be alkyl of C1-C2, cycloalkyl of C5-C6, styrene, phenyl, benzyl, or —CN.

In addition, the organic dots of the present invention may be characterized in the compound of Formula 1 or the compound of Formula 2, surface treated with polyethylenimine (PEI) or aminopolystyrene (APS). In this case, luminous efficacy, light stability, dispersibility, etc. may be enhanced.

Hereinafter a compensation film composition and a compensation film using the organic dots described above will be explained.

The compensation film composition of the present invention may include a binder; and a luminescent material, and may further include beads.

In the compensation film composition of the present invention, the binder may use at least one selected from an aliphatic urethane acrylate resin, an epoxy acrylate resin, a melamine acrylate resin, or a polyester acrylate resin, and may preferably use a thermosetting aliphatic urethane acrylate having a weight average molecular weight of 1,000-10,000. In an embodiment, the aliphatic urethane acrylate may be an acrylate obtained via the first synthesis so as to include an isocyanate group with a hydroxyl group at the terminal thereof by reacting an aliphatic polyol and a diisocyanate. For example, the aliphatic urethane acrylate may be prepared by reacting ethylhydroxy acrylate and an isocyanate, or may be commercially purchased.

In addition, the luminescent material may include the organic dots represented by Formula 1 and/or the organic dots represented by Formula 2, explained above. In this case, the amount used of the luminescent material may be 0.05-7 parts by weight, preferably, 0.07-5 parts by weight, and more preferably, 0.07-3 parts by weight relative to 100 parts by weight of the binder. In the case that the amount used of the luminescent material is less than 0.05 parts by weight, sufficient color reproducibility may not be obtained, and in the case that the amount used is greater than 7 parts by weight, transmittance may be deteriorated, and luminance may decrease. Accordingly, the amount of the luminescent material is preferably in the above-described range.

In addition, in order to manufacture a compensation film having white, that, is, white light under a blue light source, a mixture including the organic dots represented by Formula 1 and the organic dots represented by Formula 2 in a ratio of 1:0.05-20, and preferably, 1:0.1-10 by weight may be used as a luminescent material. In the case that the weight ratio is less than 1:0.05 or greater than 1:20, an x coordinate range may deviate 0.20-0.50, or a y coordinate range may deviate from 0.15-0.40 on the basis of an NTSC color coordinate of FIG. 3, and a desired compensation film may not be manufactured.

In the compensation film composition of the present invention, the beads play the role of uniformly distributing light and improving the feeling of color and may include at least one selected from mono dispersive beads or poly dispersive beads. In addition, the beads may use at least one selected from silica, zirconia, titanium dioxide, polystyrene, polypropylene, polyethylene, polyurethane or polymethyl(meth)acrylate, preferably, at least one of mono dispersive silica, polystyrene, or titanium oxide, and more preferably, mono dispersive beads including silica which is a transparent material. In addition, the amount used of the beads may be 30-1,700 parts by weight, and more preferably, 50-1,000 parts by weight relative to 100 parts by weight of a binder. In the case that the amount used of the beads is less than 30 parts by weight, light may not be uniformly distributed and the feeling of color may be deteriorated. In the case that the amount used of the beads is greater than 1,700 parts by weight, luminance may decrease. Accordingly, the amount is preferably in the above-described range. In addition, in the present invention, the average particle diameter of the beads may be 0.5-30 μm, and preferably, 0.5-10 μm. In the case that the average particle diameter of the beads is less than 0.5 μm, transmittance may decrease, and in the case that the average particle diameter of the beads is greater than 30 μm, light absorbance may decrease. Accordingly, the average particle diameter is preferably in the above-described range.

The compensation film composition of the present invention may further include a solvent as well as the binder, the luminescent material and the beads. In this case, the solvent may be at least one selected from alcohols including at least one selected from methanol, ethanol, propanol or isopropanol; ketones including at least one selected from methyl ethyl ketone or methyl isobutyl ketone; esters including at least one selected from methyl acetate or ethyl acetate; an aromatic compound including at least one selected from toluene or benzene xylene; or ethers. Preferably, a mixture of the ketones and the aromatic compound may be used in consideration of the solubility of an organic material and an advantageous drying process, however, embodiments are not limited thereto. The amount used of the solvent may be 30-200 parts by weight, and preferably, 80-120 parts by weight relative to 100 parts by weight of the binder. In the case that the amount used of the solvent is less than 30 parts by weight, the viscosity of the composition may become too high, and processability may be deteriorated, and in the case that the amount is greater than 200 parts by weight, the viscosity may become too low, drying time may be too long, and moldability may be deteriorated.

In addition, the compensation film composition of the present invention may be prepared by additionally using at least one selected from quantum dots, polymer dots or a dye other than the binder, the luminescent material, and the beads.

In this case, the quantum dots may be any one commonly used in this art, without specific limitation.

In addition, the polymer dots may include at least one selected from a random copolymer represented by the following Formula 3 or a random copolymer represented by the following Formula 4.

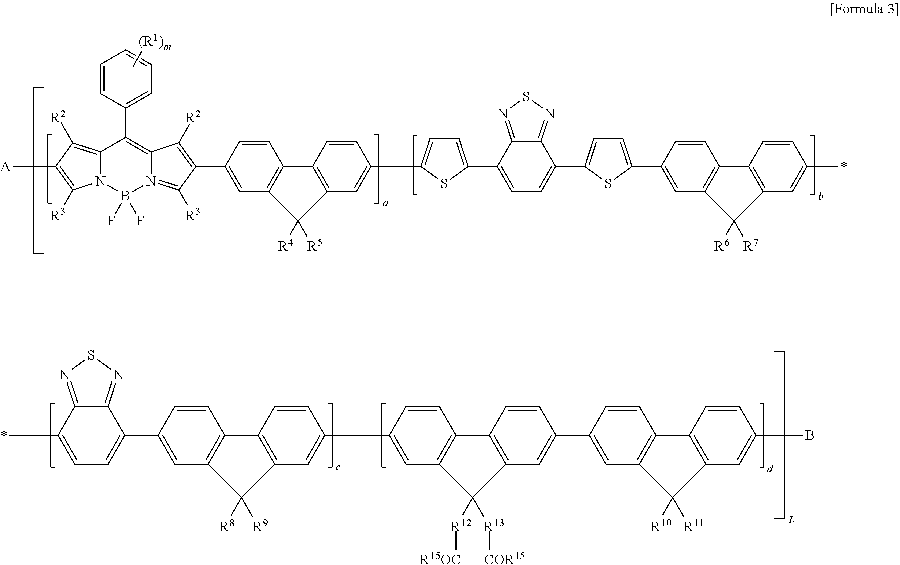

In Formula 3, R² is methyl or ethyl, m is an integer of 0-3, R² is hydrogen, methyl or ethyl, R³ is alkyl of C1-C5, olefin of C2-C5, cycloalkyl of C5-C6, olefin of C2-C4 including phenyl or

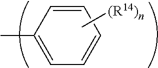

(where R¹⁴ is methyl or ethyl, and n is an integer of 0-3), R⁶-R¹¹ are each independently linear alkyl of C1-C12, branched alkyl of C4-C12, or olefin of C2-C12, R¹²-R¹³ are each independently alkyl of C1-C5, R¹⁵ is —OH, —OCH₃, or —OCH₂CH₃, a, b, c, and d represent molar ratios of monomers composing a polymer, where the molar ratio of a, b, c, and d is 1:1-1.5:5-25:1-1.5, A and B are each independently at least one terminal group selected from phenyl, phenyl, biphenyl, anthracene, or naphthalene, and L is a rational number satisfying the weight average molecular weight of a copolymer of 1,000-50,000.

In addition, preferably in Formula 3, R¹ is methyl, m is an integer of 1-3, R² is hydrogen or methyl, R³ is olefin of C1-C5 or olefin of C2-C4 including

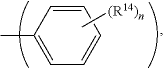

R¹⁴ is methyl, n is 0 or 1, R⁶-R¹¹ are each independently the same, R⁶-R¹¹ are linear alkyl of C6-C10 or branched alkyl of C6-C10, and A and B are phenyl.

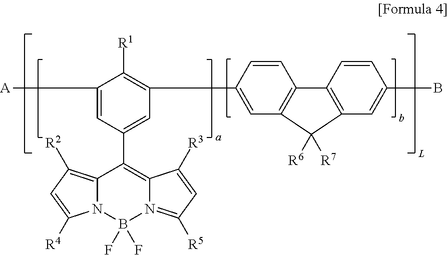

In Formula 4, R¹ is hydrogen or alkyl of C1-C5, R² and R³ are each independently hydrogen, methyl, or ethyl, R⁴ and R⁵ are each independently hydrogen, alkyl of C1-C5, olefin of C2-C5, cycloalkyl of C5-C6, olefin including phenyl or

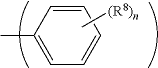

(where R⁸ is methyl or ethyl, and n is an integer of 0-3), R⁶ and R⁷ are each independently linear alkyl of C1-C12, branched alkyl of C4-C12, or olefin of C2-C12, the molar ratio of a and b is 1:5-15, A and B are each independently at least one terminal group selected from phenyl, biphenyl, anthracene, or naphthalene, and L is a rational number satisfying the weight average molecular weight of a copolymer of 1,000-100,000.

Preferably, in Formula 4, R¹ is methyl, R² and R³ are each independently hydrogen or alkyl of C1-C2, R⁴ and R⁵ are each independently hydrogen, or alkyl of C1-C5, R⁶ and R⁷ are each independently linear alkyl of C6-C10, or branched alkyl of C6-C10, and A and B are phenyl.

In addition, the dye may be a dye for an optical film used in the art, and preferably, include at least one selected from coumarin (green) or rhodamin (red).

In addition, the compensation film composition described above may further include at least one additive selected from a light stabilizer, an ultraviolet absorbent, an antistatic agent, a lubricant, a leveling enhancer, a defoamer, a polymerization accelerator, an antioxidant, a flame retardant, an infrared absorbent, a surfactant, a surface modifier, etc.

A compensation film may be manufactured using the above-described compensation film composition by a common method used in the art. For example, a final organic dot layer may be formed by coating at least one side of a base with the above-described various types of compensation film composition as a coating solution via a common method used in the art such as a meyer bar method and a comma coater method, drying and curing.

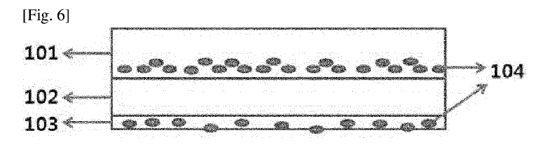

In an embodiment, a luminance improving film (or sheet) having a shape of FIG. 6 may be manufactured using the compensation film of the present invention having a shape in FIG. 5. The luminance improving film includes the compensation film of the present invention as a compensation film layer (101), and the compensation film layer may include the organic dots of the present invention described above and/or beads (104). The compensation film layer (101) may be formed on the top surface of the base (102). In addition, the base is not specifically limited, however a polyethylene terephthalate (PET) material may be used. In addition, on the bottom surface of the base (102, or a base layer), a bead coating layer (103) may be formed, and the bead coating layer (103) may include beads the same as or different from the beads (104). In addition, The compensation film and/or the luminance improving film may be used as the optical film of a back light unit, and particularly, may be formed between a light guide sheet (or a light guide plate, or a light guide film) and a prism sheet (or a film), thereby increasing the efficiency, the luminance, etc. and improving the color reproducibility of an LCD, etc.

The average thickness of the compensation film of the present invention may be 0.1-200 μm, preferably, 2-100 μm, and more preferably, 2-70 μm. In the case that the average thickness of the compensation film is less than 0.1 μm, the accomplishing of white light may become difficult, and in the case that the thickness is greater than 200 μm, the transmittance of light may be too low, and the luminance and the color reproducibility thereof may decrease. Accordingly, the thickness is preferably in the above-described range.

In addition, the base may be any material used as the base of the conventional optical film, without specific limitation and may include, for example, a polyester film and a polyethylene film such as polyethylene terephthalate, polybutylene terephthalate, polyethylene naphthalate, etc., a polypropylene film, cellophane, a diacetyl cellulose film, a triacetyl cellulose film, an acetyl cellulose butyrate film, a polyvinyl chloride film, a polyvinylidene chloride film, a polyvinyl alcohol film, an ethylene-vinyl acetate copolymer film, a polystyrene film, a polycarbonate film, a polymethylpentene film, a polysulfone film, a polyether ether ketone film, a polyether sulfone film, a polyetherimide film, a polyimide film, a fluorine resin film, a polyamide film, an acryl resin film, a norbornene resin film, a cycloolefin resin film, etc.

In addition, in the bead coating layer (103), a coating solution including a thermosetting polyurethane resin may further include an antistatic agent for obtaining antistatic effects. In this case, the antistatic agent used may be a quaternary ammonium salt-based, polymer-based antistatic agent, and the polymer-based antistatic agent may be used in an amount of 20 parts by weight or less, preferably, 3-12 parts by weight, and more preferably, 5-9 parts by weight relative to 100 parts by weight of the coating solution. Particular examples of the polymer-based antistatic agent may include ELECON-100ED, and ELECON-1700 of Nano Chem Tech Co., MORESTAT ES-7205, and MORESTAT ES-7500 of Morechem Co., JISTAT 2000/2000N of Joogil Oil Chemical Co., PU 101 of Jeil industrial pharma Co. in Japan, etc. In addition, the bead coating layer may further include a light stabilizer added for the UV stability of a coating solution for a diffusion film and a passivation film, and the examples of the light stabilizer may include Tinuvin 144, Tinuvin 292, Tinuvin 327, Tinuvin 329, Tinuvin 5050, Tinuvin 5151, etc., of Ciba Geigy Co., and LOWILITE 22, LOWILITE 26, LOWILITE 55, LOWILITE 62, LOWILITE 94, etc. of Miwon Commercial Co., etc., and the present invention is not limited thereto.

In addition, the bead coating layer may be manufactured by appropriately adding at least one additive of an ultraviolet absorbent, a lubricant, a leveling agent, a defoamer, a polymerization accelerator, an antioxidant, a flame retardant, an infrared absorbent, a surfactant, a surface modifier, etc.

The compensation film including the organic dots of the present invention as explained above may be widely used by applying to a light emitting diode (LED) display, a light emitting diode (LED) illumination apparatus and/or a liquid crystal display (LCD), etc. For example, the present invention relates to a novel material for improving color reproducibility, luminance, etc. regarding red (R), and green (G) by applying to a prism film, a diffusion film, a light guide plate, a compensation film, or a reflection polarizer in a backlight unit (BLUs). The present invention may be very appropriately used in a compensation film for an LCD, a reflection polarizer, etc.

In addition, the organic dots of the present invention are a luminescent material not using an inorganic material such as cadmium and do not induce environmental issues. The organic dots represented by Formula 1 have a PL wavelength of 580-680 nm, and decreasing half width (color reproducibility) of a red system and increasing quantum efficiency (luminous efficacy). In addition, the organic dots represented by Formula 2 according to the present invention have a wide PL wavelength of 500-680 nm, and decreasing half width (color reproducibility) of a green system and increasing quantum efficiency (luminous efficacy). In addition, the organic dots represented by Formula 1 and/or the organic dots represented by Formula 2 may be used per se or as an application type such as a compensation film, and may be used in various fields including a bio sensor, an illumination apparatus, a display, etc.

Hereinafter the present invention will be explained in more detail referring to embodiments. However, the scope of the present invention is not limited by the following embodiments.

### EXAMPLES

### Example 1

### Preparation of Organic Dots Represented by Formula 1-1

To a three-necked flask, 1.0 g (1.199 mmol) of Formula a, and 828 mg (5.995 mmol) of K₂CO₃ were added, followed by evacuating. Nitrogen was injected, and n-methyl-2-pyrrolidone (NMP) was added thereto, followed by stirring.

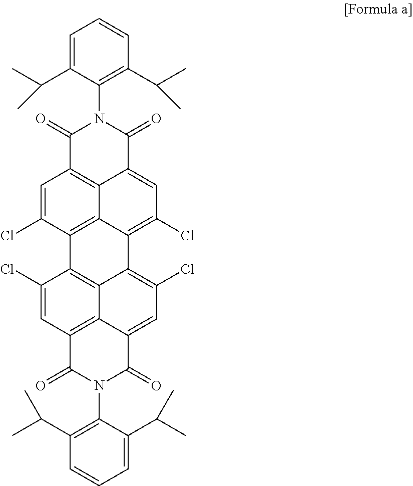 [SMILES: CC(C)c1cccc(C(C)C)c1N1C(=O)c2cc(Cl)c3c4c(Cl)cc5c6c(cc(Cl)c(c7c(Cl)cc(c2c37)C1=O)c64)C(=O)N(c1c(C(C)C)cccc1C(C)C)C5=O]

Then, 564 mg (5.995 mmol) of phenol was added thereto, followed by heating to 80° C. and stirring at the temperature for 15 minutes to finish the reaction.

The reaction product was treated with water and an MgSO₄ solution to capture water, and dried using a rotary evaporator. After that, the dried reaction product was separated using column chromatography to obtain a compound represented by the following Formula 1-1.

¹H NMR (CDCl₃, 400 MHz): 7.543 (t, 8H), 7.443 (t, 2H), 7.284 (m, 8H), 7.159 (t, 4H), 7.097 (d, 8H), 2.953 (m, 4H), 1.617 (d, 24H)

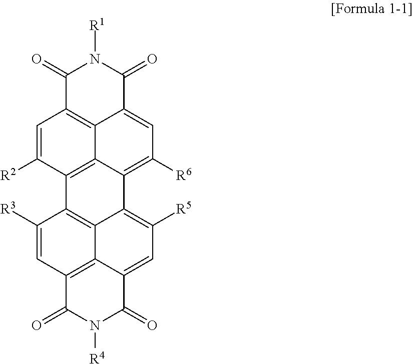

In Formula 1, R² and R⁴ are

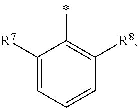

R⁷ and R⁸ are isopropyl, and R², R³, R⁵ and R⁶ are phenoxy.

### Example 2

### Preparation of Organic Dots Represented by Formula 1-2

To a three-necked flask, 1.0 g (0.927 mmol) of a compound represented by Formula 1-1 and prepared in Example 1, and 5 ml of H₂SO₄ were added, followed by stirring at room temperature for 15 hours to finish the reaction. Then, the reaction product was injected to water slowly, and solid was filtered.

Then, the solid thus filtered was washed with dichloromethane about three times, dried at 100° C. in vacuum to obtain a compound represented by Formula 1-2.

¹H NMR (CD₃OD, 400 MHz): 8.183 (s, 4H), 7.877 (d, 8H), 7.447 (t, 2H), 7.325 (d, 4H), 7.168 (d, 8H), 2.725 (m, 4H), 1.131 (d, 24H)

[Formula 1-2]

In Formula 1, R¹ and R⁴ are

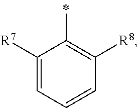

R⁷ and R⁸ are isopropyl, R², R³, R⁵ and R⁶ are

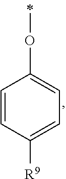

and R⁹ is —SO₃H.

### Example 3

### Preparation of Organic Dots Represented by Formula 1-3

To a three-necked flask, 1.0 g (1.199 mmol) of Formula a, and 828 mg (5.995 mmol) of K₂CO₃ were added, followed by evacuating. Nitrogen was injected, and n-methyl-2-pyrrolidone (NMP) was added thereto, followed by stirring.

Then, 996 mg (5.995 mmol) of methyl(4-hydroxyphenyl)acetate was added thereto, followed by heating to 60° C. and stirring at the temperature for 15 minutes to finish the reaction.

The reaction product was cooled to 25° C., and hydrochloric acid was injected thereto. Then, the pH of the reaction product was controlled to neutral using water, followed by washing and drying in vacuum. After that, the dried reaction product was separated using column chromatography to obtain a compound represented by the following Formula 1-3.

¹H NMR(C₂D₂Cl₄, 400 MHz): 8.147 (s, 4H), 7.882 (d, 8H), 7.342 (t, 2H), 7.189 (d, 4H), 7.097 (d, 8H), 3.802 (s, 8H), 2.497 (m, 4H), 1.061 (d, 24H)

[Formula 1-3]

In Formula 1, R² and R⁴ are

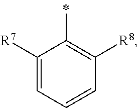

R⁷ and R⁸ are isopropyl, R², R³, R⁵ and R⁶ are

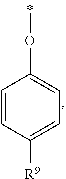

and R⁹ is —CH₂CH₂COOH.

### Example 4

### Preparation of Organic Dots Represented by Formula 1-4

To a three-necked flask, 1.0 g (1.199 mmol) of Formula a, 828 mg (5.995 mmol) of K₂CO₃, and 990 mg (5.995 mmol) of Hordenine were added, followed by evacuating. Nitrogen was injected, and NMP was added thereto, followed by stirring.

Then, the reactant was heated to 100° C. and stirred at the temperature for 15 minutes to finish the reaction.

The reaction product was cooled to 25° C., hydrochloric acid was injected thereto, solid was filtered, and the solid thus filtered was washed with water. After that, the solid thus washed was dried in vacuum, and the dried solid was separated using column chromatography to obtain a compound represented by the following Formula 1-4.

¹H NMR (CDCl₃, 400 MHz): 8.165 (s, 4H), 7.447 (t, 2H), 7.312 (d, 4H), 7.308 (d, 8H), 7.012 (d, 8H), 2.848 (m, 12H), 2.470 (m, 8H), 2.248 (s, 24H), 1.077 (d, 24H)

[Formula 1-4]

In Formula 1, R¹ and R⁴ are

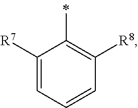

R⁷ and R⁸ are

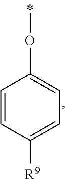

R⁹ is —CH₂NR¹¹R¹², and R¹¹ and R¹² are methyl.

### Example 5

### Preparation of Organic Dots Represented by Formula 1-5

To a three-necked flask, 1.0 g (1.199 mmol) of Formula a, 828 mg (5.995 mmol) of K₂CO₃, and 912 mg (9.592 mmol) of 3-hydroxypyridine were added, followed by evacuating. Nitrogen was injected, and NMP was added thereto, followed by stirring.

Then, the reactant was heated to 100° C. and stirred at the temperature for 15 minutes to finish the reaction.

The reaction product was cooled to 25° C., hydrochloric acid was injected thereto, solid was filtered, and the solid thus filtered was washed with water. After that, the solid thus washed was dried in vacuum, and the dried reaction product was separated using column chromatography to obtain a compound represented by the following Formula 1-5.

¹H NMR (C₂D₂Cl₄, 400 MHz): 8.287 (d, 4H), 8.279 (s, 4H), 8.138 (s, 4H), 7.348 (t, 2H), 7.286 (m, 4H), 7.179 (d, 4H), 7.182 (d, 4H), 2.577 (m, 4H), 1.037 (d, 24H)

[Formula 1-5]

In Formula 1, R¹ and R⁴ are

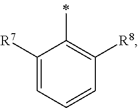

R⁷ and R⁸ are isopropyl, and R², R³, R⁵ and R⁶ are

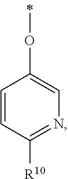

and R¹⁰ is hydrogen.

### Example 6

### Preparation of Organic Dots Represented by Formula 1-6

To a three-necked flask, 1.0 g (1.151 mmol) of Formula b, and 795 mg (5.755 mmol) of K₂CO₃ were added, followed by evacuating. Nitrogen was injected, and NMP was added thereto, followed by stirring.

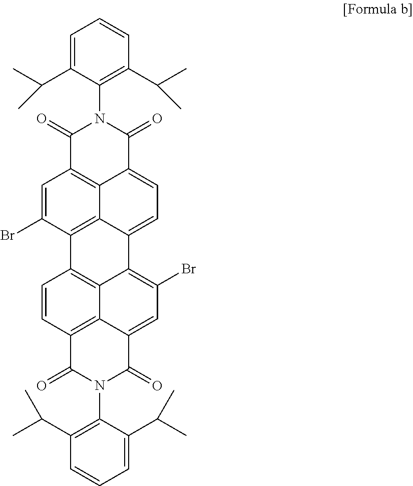 [SMILES: CC(C)c1cccc(C(C)C)c1N1C(=O)c2ccc3c4c(Br)cc5c6c(ccc(c7c(Br)cc(c2c37)C1=O)c64)C(=O)N(c1c(C(C)C)cccc1C(C)C)C5=O]

Then, 541 mg (5.755 mmol) of phenol was added thereto, followed by heating to 100° C. and stirring at the temperature for 15 minutes to finish the reaction.

The reaction product was cooled to 25° C., hydrochloric acid was injected thereto, solid was filtered, and the solid thus filtered was washed with water. After that, the solid thus washed was dried in vacuum, and the dried reaction product was separated using column chromatography to obtain a compound represented by the following Formula 1-6.

¹H NMR (CDCl₃, 400 MHz): 9.554 (d, 2H), 8.548 (d, 2H), 8.283 (s, 2H), 7.423 (m, 6H), 7.233 (m, 10H), 2.601 (m, 4H), 1.053 (m, 24H)

[Formula 1-6]

In Formula 1, R² and R⁴ are

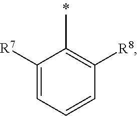

R⁷ and R⁸ are isopropyl, and R² and R⁵ are

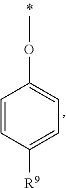

R⁹ is hydrogen, and R³ and R⁶ are hydrogen.

### Example 7

### Preparation of Organic Dots Represented by Formula 1-7

To a three-necked flask, 1.0 g (1.117 mmol) of the compound represented by Formula 1-6, which was prepared in Example 6, and 5 ml of H₂SO₄ were injected, followed by stirring at 25° C. for 15 hours to finish the reaction.

Then, water was injected slowly to the reaction product, and solid was filtered. Then, the solid thus filtered was washed with dichloromethane about three times, dried at 100° C. in vacuum to obtain a compound represented by Formula 1-7.

¹H NMR (CD₃OD, 400 MHz): 8.874 (d, 2H), 8.167 (d, 2H), 8.014 (s, 2H), 7.541 (d, 4H), 7.163 (t, 2H), 7.043 (d, 4H), 6.934 (d, 4H), 2.438 (m, 4H), 0.871 (m, 24H)

[Formula 1-7]

In Formula 1, R¹ and R⁴ are

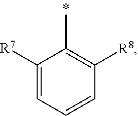

R⁷ and R⁸ are isopropyl, and R² and R⁵ are

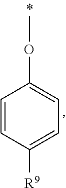

R⁹ is —SO₃H, and R³ and R⁶ are hydrogen.

### Example 8

### Preparation of Organic Dots Represented by Formula 2-1

To a three-necked flask, 0.59 ml (4 mmol) of 2,4,6-trimethylbenzaldehyde was added, followed by evacuating. Dried CH₂Cl₂ was added thereto, followed by stirring.

Then, 1.029 ml (10 mmol) of 2,4-dimethyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 0.90 g (4 mmol) of 2,3-dichloro-5,6-dicyano-1,4-benzoquinone was injected thereto at 0° C., followed by elevating the temperature to 25° C. and stirring for 1 hour.

Then, 8.1 ml (57.6 mmol) of triethylamine (NEt₃) was injected, and 8.6 ml (68 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-1.

¹H NMR (CDCl₃, 400 MHz): 6.967 (s, 2H), 5.983 (s, 2H), 2.579 (s, 6H), 2.355 (s, 3H), 2.114 (s, 6H), 1.402 (s, 6H)

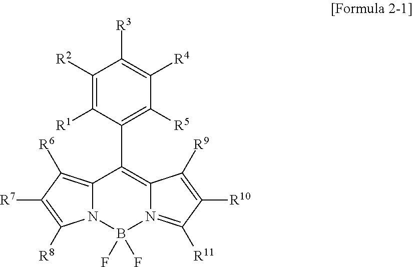

In Formula 2-1, R², R⁴, R⁷ and R¹⁰ are hydrogen, and R¹, R³, R⁵, R⁶, R⁸, R⁹ and R¹¹ are alkyl of C1.

### Example 9

### Preparation of Organic Dots Represented by Formula 2-2

To a three-necked flask, 1.0 g (6.747 mmol) of 2,4,6-trimethylbenzaldehyde was added, followed by evacuating. Dried CH₂Cl₂ was added thereto, followed by stirring.

Then, 1.37 g (16.869 mmol) of 2-methyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.54 g (6.747 mmol) of 2,3-dichloro-5,6-dicyano-1,4-benzoquinone was injected thereto at 0° C., followed by elevating the temperature to 25° C. and stirring for 1 hour.

Then, 13 ml (97.156 mmol) of triethylamine (NEt₃) was injected, and 14 ml (114.699 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-2.

¹H NMR (CDCl₃, 400 MHz): 6.86 (s, 2H), 5.82 (d, 2H), 2.60 (s, 6H), 2.33 (s, 3H), 2.12 (s, 6H), 1.41 (d, 2H)

[Formula 2-2]

In Formula 2, R², R⁴, R⁶, R⁷, R⁹, and R¹⁰ are hydrogen, and R⁴, R³, R⁵, R⁸, and R¹¹ are alkyl of C1.

### Example 10

### Preparation of Organic Dots Represented by Formula 2-3

To a three-necked flask, 1.0 g (6.747 mmol) of 2,4,6-trimethylbenzaldehyde was added, followed by evacuating. Dried CH₂Cl₂ was added thereto, followed by stirring.

Then, 1.37 g (16.869 mmol) of 2-methyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.54 g (6.747 mmol) of 2,3-dichloro-5,6-dicyano-1,4-benzoquinone was injected thereto at 0° C., followed by elevating the temperature to 25° C. and stirring for 1 hour to finish the reaction.

Then, 13 ml (97.156 mmol) of triethylamine (NEt₃) was injected, and 14 ml (114.699 mmol) of BF₃.Et₂O were slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-3.

¹H NMR (CDCl₃, 400 MHz): 6.87 (s, 2H), 6.99 (d, 2H), 5.78 (d, 2H), 2.36 (s, 3H), 2.14 (s, 6H), 1.46 (s, 2H)

[Formula 2-3]

In Formula 2, R², R⁴, R⁷, R⁸, R¹⁰, and R¹¹ are hydrogen, and R², R³, R⁶, and R⁹ are alkyl of C1.

### Example 11

### Preparation of Organic Dots Represented by Formula 2-4

To a three-necked flask, 1.0 g (6.246 mmol) of 2,4,6-trifluorobenzaldehyde was added, followed by evacuating. Dried CH₂Cl₂ was added thereto, followed by stirring.

Then, 1.48 g (15.615 mmol) of 2,4-dimethyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.42 g (6.246 mmol) of 2,3-dichloro-5,6-dicyano-1,4-benzoquinone was injected thereto at 0° C., followed by elevating the temperature to 25° C. and stirring for 1 hour.

Then, 12.0 ml (89.942 mmol) of triethylamine (NEt₃) was injected, and 13.0 ml (106.182 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-4.

¹H NMR (CDCl₃, 400 MHz): 6.40 (s, 2H), 5.84 (s, 2H), 2.72 (s, 6H), 1.49 (s, 6H)

[Formula 2-4]

In Formula 2, R², R⁴, R⁷, and R¹⁰ are hydrogen, and R¹, R³, and R⁵ are fluorine, and R⁶, R⁸, R⁹ and R¹¹ are alkyl of C1.

### Example 12

### Preparation of Organic Dots Represented by Formula 2-5

To a three-necked flask, 1.0 g (7.626 mmol) of 4-formylbenzonitrile was added, followed by evacuating. Dried CH₂Cl₂ was added thereto, followed by stirring.

Then, 1.80 g (19.065 mmol) of 2,4-dimethyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.73 g (7.626 mmol) of 2,3-dichloro-5,6-dicyano-1,4-benzoquinone was injected thereto at 0° C., followed by elevating the temperature to 25° C. and stirring for 1 hour.

Then, 15.0 ml (109.814 mmol) of triethylamine (NEt₃) was injected, and 16.0 ml (129.642 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-5.

¹H NMR (CDCl₃, 400 MHz): 7.87 (d, 2H), 7.56 (d, 2H), 5.75 (s, 2H), 2.67 (s, 6H), 1.45 (s, 6H)

[Formula 2-5]

In Formula 2, R¹, R², R⁴, R⁵, R⁷, and R¹⁰ are hydrogen, and R³ is —CN, and R⁶, R⁸, R⁹ and R¹¹ are alkyl of C1.

### Example 13

### Preparation of Organic Dots Represented by Formula 2-6

To a three-necked flask, 1.0 g (7.626 mmol) of 3-formylbenzonitrile was added, followed by evacuating. Dried CH₂Cl₂ was added thereto, followed by stirring.

Then, 1.80 g (19.065 mmol) of 2,4-dimethyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.73 g (7.626 mmol) of 2,3-dichloro-5,6-dicyano-1,4-benzoquinone was injected thereto at 0° C., followed by elevating the temperature to 25° C. and stirring for 1 hour.

Then, 15.0 ml (109.814 mmol) of triethylamine (NEt₃) was injected, and 16.0 ml (129.642 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-6.

¹H NMR (CDCl₃, 400 MHz): 7.61-7.84 (m, 4H), 5.73 (s, 2H), 2.69 (s, 6H), 1.47 (s, 6H)

[Formula 2-6]

In Formula 2, R¹, R², R³, R⁵, R⁷, and R¹⁰ are hydrogen, and R⁴ is —CN, and R⁶, R⁸, R⁹ and R¹¹ are alkyl of C1.

### Example 14

### Preparation of Organic Dots Represented by Formula 2-7

To a three-necked flask, 1.0 g (5.984 mmol) of 3,5-difluoro-4-formylbenzonitrile was added, followed by evacuating. Dried CH₂Cl₂ was added thereto, followed by stirring.

Then, 1.42 g (14.960 mmol) of 2,4-dimethyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.36 g (5.984 mmol) of 2,3-dichloro-5,6-dicyano-1,4-benzoquinone was injected thereto at 0° C., followed by elevating the temperature to 25° C. and stirring for 1 hour.

Then, 12.0 ml (86.169 mmol) of triethylamine (NEt₃) was injected, and 13.0 ml (101.728 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-7.

¹H NMR (CDCl₃, 400 MHz): 6.94 (s, 2H), 5.80 (s, 2H), 2.70 (s, 6H), 1.49 (s, 6H)

[Formula 2-7]

In Formula 2, R², R⁴, R⁷, and R¹⁰ are hydrogen, R¹ and R⁵ are fluorine, R³ is —CN, and R⁶, R⁸, R⁹ and R¹¹ are alkyl of C1.

### Example 15

### Preparation of Organic Dots Represented by Formula 2-8

To a three-necked flask, 1.0 g (6.747 mmol) of 2,4,6-trimethylbenzaldehyde was added, followed by evacuating. Dried CH₂Cl₂ was added thereto, followed by stirring.

Then, 1.79 g (16.869 mmol) of 4-methyl-1H-pyrrole-2-carbontrile was added thereto, and trifluoroacetic acid (44 UI) and dried CH₂Cl₂ were diluted and added thereto slowly.

After that, the reaction mixture was stirred at 25° C. for 3 hours, and 1.54 g (6.747 mmol) of 2,3-dichloro-5,6-dicyano-1,4-benzoquinone was injected thereto at 0° C., followed by elevating the temperature to 25° C. and stirring for 1 hour.

Then, 13 ml (97.156 mmol) of triethylamine (NEt₃) was injected, and 14 ml (114.699 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-8.

¹H NMR (CDCl₃, 400 MHz): 6.90 (s, 2H), 5.92 (s, 2H), 2.34 (s, 3H), 2.10 (s, 6H), 1.40 (s, 6H)

[Formula 2-8]

R⁷ and R¹⁰ are hydrogen, and R⁸ and R¹¹ are —CN.

### Example 16

### Preparation of Organic Dots Represented by Formula 2-9

To a three-necked flask, 1.0 g (6.747 mmol) of 2,4,6-trimethylbenzaldehyde was added, followed by evacuating. Dried CH₂Cl₂ was added thereto, followed by stirring.

Then, 2.65 g (16.869 mmol) of 2-methyl-4-phenylpyrrole was added thereto, and trifluoroacetic acid (44 UI) and dried CH₂Cl₂ were diluted and added thereto slowly.

After that, the reaction mixture was stirred at 25° C. for 3 hours, and 1.54 g (6.747 mmol) of 2,3-dichloro-5,6-dicyano-1,4-benzoquinone was injected thereto at 0° C., followed by elevating the temperature to 25° C. and stirring for 1 hour.

Then, 13 ml (97.156 mmol) of triethylamine (NEt₃) was injected, and 14 ml (114.699 mmol) of BF₃.Et₂O was slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-9.

¹H NMR (CDCl₃, 400 MHz): 7.29-7.50 (m, 10H), 6.88 (s, 2H), 5.89 (s, 2H), 2.56 (s, 6H), 2.32 (s, 3H), 2.11 (s, 6H)

[Formula 2-9]

In Formula 2, R¹, R³, R⁵, R⁸ and R¹¹ are methyl, R², R⁴, R⁷ and R¹⁰ are hydrogen, and R⁶ and R⁹ are phenyl.

### Example 17

### Preparation of Organic Dots Represented by Formula 2-10

To a three-necked flask, 1.0 g (6.747 mmol) of 2,4,6-trimethylbenzaldehyde was added, followed by evacuating. Dried CH₂Cl₂ was added thereto, followed by stirring.

Then, 2.94 g (16.869 mmol) of 4-benzyl-2-methyl-1H-pyrrole was added thereto, and trifluoroacetic acid (44 UI) and dried CH₂Cl₂ were diluted and added thereto slowly.

After that, the reaction mixture was stirred at 25° C. for 3 hours, and 1.54 g (6.747 mmol) of 2,3-dichloro-5,6-dicyano-1,4-benzoquinone was injected thereto at 0° C., followed by elevating the temperature to 25° C. and stirring for 1 hour.

Then, 13 ml (97.156 mmol) of triethylamine (NEt₃) was injected, and 14 ml (114.699 mmol) of BF₃.Et₂O were slowly injected, followed by stirring at 25° C. for 5 hours to finish the reaction.

The reaction product was treated with an Na₂CO₃ solution and an Na₂SO₄ solution to capture water, and dried using a rotary evaporator. After that, the dried reaction product was separated using column chromatography to obtain a compound represented by the following Formula 2-10.

¹H NMR (CDCl₃, 400 MHz): 7.24-7.36 (m, 10H), 6.86 (s, 2H), 5.89 (s, 2H), 3.60 (s, 4H), 2.54 (s, 6H), 2.30 (s, 3H), 2.08 (s, 6H)

[Formula 2-10]

In Formula 2, R¹, R³, R⁵, R⁸ and R¹¹ are methyl, R², R⁴, R⁷ and R¹⁰ are hydrogen, and R⁶ and R⁹ are benzyl.

TABLE 1

| Division | R¹ | R² | R³ | R⁴ | R⁵ | R⁶ | R⁷ | R⁸ | R⁹ | R¹⁰ | R¹¹ |
|---|---|---|---|---|---|---|---|---|---|---|---|
| Example 8 | —CH₃ | —H | —CH₃ | —H | —CH₃ | —CH₃ | —H | —CH₃ | —CH₃ | —H | —CH₃ |
| Example 9 | —CH₃ | —H | —CH₃ | —H | —CH₃ | —H | —H | —CH₃ | —H | —H | —CH₃ |
| Example 10 | —CH₃ | —H | —CH₃ | —H | —CH₃ | —CH₃ | —H | —H | —CH₃ | —H | —H |
| Example 11 | —F | —H | —F | —H | —F | —CH₃ | —H | —CH₃ | —CH₃ | —H | —CH₃ |
| Example 12 | —H | —H | —CN | —H | —H | —CH₃ | —H | —CH₃ | —CH₃ | —H | —CH₃ |
| Example 13 | —H | —H | —H | —CN | —H | —CH₃ | —H | —CH₃ | —CH₃ | —H | —CH₃ |
| Example 14 | —F | —H | —CN | —H | —F | —CH₃ | —H | —CH₃ | —CH₃ | —H | —CH₃ |
| Example 15 | —CH₃ | —H | —CH₃ | —H | —CH₃ | —CH₃ | —H | —CN | —CH₃ | —H | —CN |
| Example 16 | —CH₃ | —H | —CH₃ | —H | —CH₃ | Phenyl | —H | —CH₃ | Phenyl | —H | —CH₃ |
| Example 17 | —CH₃ | —H | —CH₃ | —H | —CH₃ | benzyl | —H | —CH₃ | benzyl | —H | —CH₃ |

### Preparation Example 1

### Manufacture of Compensation Film

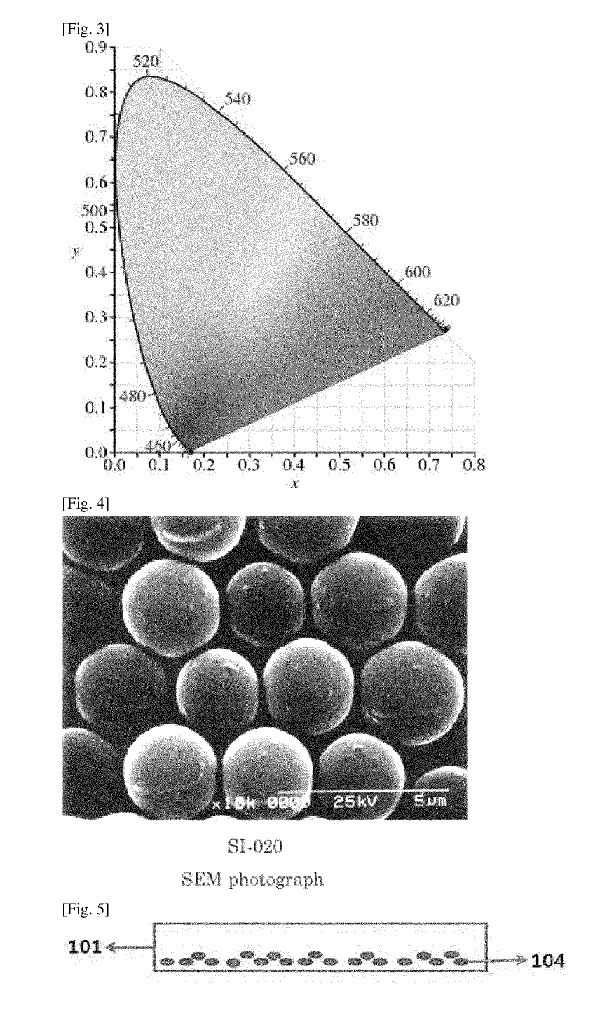

Relative to 100 parts by weight of a two-component type thermosetting urethane resin, which has a weight average molecular weight of 2,000 and six functional groups, 500 parts by weight of silicon mono dispersive beads having an average particle diameter of 2 μm and the shape shown in FIG. 4 (SI-020 of Gans Co.), 120 parts by weight of methyl ethyl ketone (MEK) and 80 parts by weight of toluene as solvents, 1 part by weight of a leveling enhancer [BYK-377, BYK Chemie Co.], 9 parts by weight of a quaternary ammonium salt-based antistatic agent (Jeil industrial pharma Co. in Japan, PU101), and 0.1 parts by weight of organic dots prepared in Example 1, as a luminescent material were mixed, followed by stirring at 1,000 rpm for 30 minutes to prepare a coating composition for manufacturing a compensation film.

The coating composition for manufacturing a compensation film was coated on the top surface of a base (PET) by a gravure coating method to an average coating thickness of 50 μm. Then, the base with the coating layer formed thereon was injected to an oven and cured at 100° C. for 10 minutes to manufacture a compensation film.

### Preparation Examples 2-7

According to Preparation Examples 2-7, compensation films were manufactured by conducting the same procedure described in Preparation Example 1 using the organic dots of Examples 2-7, respectively.

### Preparation Example 8

### Surface Treatment of Organic Dots and Manufacture of Compensation Film Using the Same

(1) The organic dots prepared in Example 8 were added to toluene and then, injected to a schlenk flask through a cannula, followed by reacting at 100° C. for about 30 minutes. Then, dean-stark for removing water and performing reaction was removed, and the flask was blocked with a stopper. The reactant was cooled to 60° C., and hexane was injected thereto through the cannula, followed by stirring. After finishing the reaction, hexane was removed through the cannula, and the reaction product was cooled to 25° C. to increase the purity of the organic dots.

(2) After connecting the schlenk flask and the dean stark, 24 g of polyethylenimine (SP-012, Nippon Shokubai Co.) was injected to the schlenk flask (250 ml), and water and oxygen were removed under 1 atm and a nitrogen atmosphere to prepare a reaction solution.

Then, 15 g of 1,2-epoxy-3-phenoxypropane (Sigma-Aldrich Co.) was injected to the reaction solution using a syringe. Then, 80 ml of toluene was injected to the schlenk flask through the cannula, followed by reacting at 100° C. for about 30 minutes. After 30 minutes, water in the dean stark was removed.

Then, 0.04 g of the surface treated organic dots were injected thereto to prepare a compensation film composition.

2) Manufacture of Compensation Film

Relative to 100 parts by weight of the organic dots prepared in Example 8, 31,500 parts by weight of an epoxy resin (Sigma-Aldrich Co., 1,2-epoxy-3-phenoxypropane), 168,000 parts by weight of a solvent (toluene), and 100 parts by weight of a dispersant for an organic material (BYK Co., Disperbyk-130) were mixed to prepare a coating composition for manufacturing a compensation film.

The coating composition for manufacturing a compensation film was coated on the top surface of a base (PET) by a gravure method to an average coating thickness of 50 μm. Then, the base with the coating layer formed thereon was injected to an oven and cured at 100° C. for 10 minutes to manufacture a compensation film.

### Preparation Examples 9-17

According to Preparation Examples 9-17, compensation films were manufactured by conducting the same procedure described in Preparation Example 8 using the organic dots of Examples 9-17, respectively.

### Experimental Example 1

### Measuring Experiments of UV Absorbance Wavelength, PL Wavelength and Luminous Efficacy

(1) Measurement of UV Absorbance Wavelength

The UV absorbance ratios of the compensation films manufactured in Preparation Examples 1-7 and the compensation films manufactured in Preparation Examples 8-17 were measured using an UV spectrometer (VARIAN, CARY 100 Conc.). The results are shown in the following Table 2.

(2) Measuring Experiment of Photoluminescence (PL)

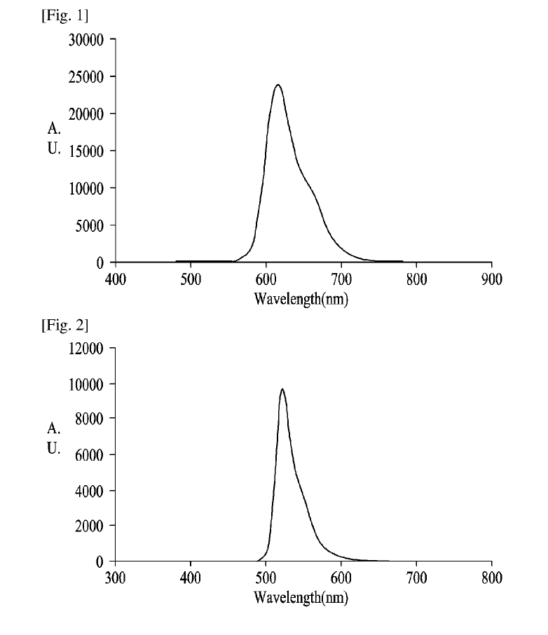

The PL measurement of each of the compensation films manufactured in Preparation Examples 1 and 8 was conducted using DarsaPro52000EM PL (PSI Trading Co.) and a 500 W ARC xenon lamp, and the results of the PL measurement are shown in FIGS. 1 and 2. As a specimen, 0.04 g of each of organic dots were taken, dissolved in 3 ml of toluene, and injected to a test tube, and emission spectrum was measured via the xenon lamp.

Referring to FIG. 1, the film manufactured in Preparation Example 1 was secured to have a peak at 618 nm, and referring to FIG. 2, the compensation film manufactured in Preparation Example 8 was secured to have a peak at 521 nm. From the results, it could be secured that the compensation film of Preparation Example 1 and the organic dots in the compensation film had the PL wavelength of a red system, and the compensation film of Preparation Example 8 and the organic dots in the compensation film had the PL wavelength of a green system

(3) Measuring Experiment of Luminous Efficacy

The luminous efficacy of the compensation films manufactured in Preparation Examples 1-8 was obtained by the following Mathematical Formula 1, and the results are shown in the following Table 3.

Q.Y.ₛₐₘₚₗₑ(luminous efficacy, %)=Q.Y._{ref}×[A_{ref}/Aₛₐₘₚₗₑ]×[n²ₛₐₘₚₗₑ/n²_{ref}]×[Dₛₐₘₚₗₑ/D_{ref}] [Mathematical Formula 1]

(A: Absorbance at 450 nm, n: refractive index of solvent, D: Integrated emission intensity)

TABLE 2

| | UV absorbance | PL wavelength | Luminous |
|---|---|---|---|
| | wavelength | measurement | efficacy |
| Division | (unit, nm) | (unit, nm) | (%) |
| Preparation | 572 | 600 | 98 |
| Example 1 | | | |
| Preparation | 460, 539, 566 | 625 | 59 |
| Example 2 | | | |
| Preparation | 529, 560 | 619 | 7 |
| Example 3 | | | |
| Preparation | 458, 548, 588 | 628 | 13 |
| Example 4 | | | |
| Preparation | 432, 519, 550 | 590 | 64 |
| Example 5 | | | |
| Preparation | 397, 498, 530 | 584 | 54 |
| Example 6 | | | |
| Preparation | 410, 520, 551 | 593 | 12 |
| Example 7 | | | |
| Preparation | 453 | 521 | 61.5 |
| Example 8 | | | |
| Preparation | 449 | 516 | 55.3 |
| Example 9 | | | |
| Preparation | 441 | 515 | 56.0 |
| Example 10 | | | |
| Preparation | 437 | 550 | 49.3 |
| Example 11 | | | |
| Preparation | 439 | 545 | 50.0 |
| Example 12 | | | |
| Preparation | 436 | 547 | 48.7 |
| Example 13 | | | |
| Preparation | 438 | 549 | 49.7 |
| Example 14 | | | |
| Preparation | 440 | 550 | 48.9 |
| Example 15 | | | |
| Preparation | 475 | 551 | 51.5 |
| Example 16 | | | |
| Preparation | 482 | 556 | 49.8 |
| Example 17 | | | |

From the measured results, the compensation films of Preparation Examples 1-7 had the PL wavelength range of 580-680 nm, and preferably, 580-640 nm. The compensation films of Preparation Examples 8-17 had the PL wavelength range of 500-680 nm, and preferably, the PL wavelength range of 510-570 nm. For the case of Preparation Examples 11-17, in which —H and/or —CN were introduced to R¹, R³ and/or R⁵ in Formula 2, the PL wavelength tended to shift toward a red direction.

In addition, the compensation films of Preparation Examples 1, 2, 5 and 6 exhibited high luminous efficacy of 50% or more, preferably, 55% or more, and more preferably, 60% or more, and the compensation films of Preparation Examples 8-17 also exhibited high luminous efficacy of 48% or more, and preferably, 55% or more.

### Preparation Example 18

A compensation film was manufactured according to the same procedure described in Example 1 except for using 0.1 parts by weight of the organic dots of Example 1 and 0.5 parts by weight of the organic dots of Example 8 as luminescent materials.

### Preparation Examples 19-23 and Comparative Preparation Examples 1-2

Compensation films of Preparation Examples 19-23 and Comparative Preparation Examples 1-2 were manufactured according to the same procedure described in Example 18 except for using the organic dots of Example 1 and the organic dots of Example 8 in amount ratios shown in the following Table 3.

### Comparative Preparation Examples 3-4

Compensation films of Comparative Examples 3-4 were manufactured according to the same procedure described in Example 18 except for using a bisphenol A epoxy diacrylate compound having a weight average molecular weight of 700 and two functional groups instead of the two-component type thermosetting urethane resin having a weight average molecular weight of 2,000 and six functional groups as a binder, and except for using the organic dots in amount ratios shown in the following Table 2.

### Comparative Preparation Example 5

A compensation film was manufactured according to the same procedure described in Example 18 except for using total 0.03 parts by weight of the organic dots prepared in Example 1 and the organic dots prepared in Example 8 relative to 100 parts by weight of the binder.

TABLE 3

| | Organic dots | | |
|---|---|---|---|
| | | | Weight |
| Division | Example 1 | Example 8 | ratio |
| Preparation Example | 0.1 parts by weight | 0.5 parts by | 1:5 |
| 18 | | weight | |
| Preparation Example | 0.1 parts by weight | 1 parts by | 1:10 |
| 19 | | weight | |
| Preparation Example | 0.1 parts by weight | 2 parts by weight | 1:20 |
| 20 | | | |
| Preparation Example | 0.5 parts by weight | 0.1 parts by | 1:0.2 |
| 21 | | weight | |
| Preparation Example | 1 parts by weight | 0.1 parts by | 1:0.1 |
| 22 | | weight | |
| Preparation Example | 2 parts by weight | 0.1 parts by | 1:0.05 |
| 23 | | weight | |
| Comparative | 0.1 parts by weight | 5 parts by weight | 1:50 |
| Preparation Example 1 | | | |
| Comparative | 5 parts by weight | 0.1 parts by | 50:1 |
| Preparation Example 2 | | weight | |
| Comparative | 0.1 parts by weight | 1 parts by weight | 1:10 |
| Preparation Example 3 | | | |
| Comparative | 1 parts by weight | 0.1 parts by | 1:0.1 |
| Preparation Example 4 | | weight | |
| Comparative | 0.01 parts by | 0.02 parts by | 1:2 |
| Preparation Example 5 | weight | weight | |

### Experimental Example 2

### Measuring Experiment of Physical Properties of Compensation Film

(1) Measuring Experiment of Color Coordinate

The measuring experiment of color coordinate was conducted using the compensation films manufactured in Preparation Examples 18-23 and Comparative Preparation Examples 1-5 by means of DarsaPro5200EM PL (PSI Trading Co.) and a 500 W ARC xenon lamp, and the results are shown in the following Table 4. The color coordinate was measured on the basis of an NTSC color coordinate shown in FIG. 3.

(2) Evaluation Method of Adhesive Strength

A specimen with 10 mm in each dimension was cross hatched by 10×10 with 1 mm unit for division. An Ichibang cellotape (18 mm, JIS Z-1522) was attached on 100 cells and pushed using hands for close attachment, and then, the tape was rapidly separated in a perpendicular direction to an attachment direction. In this case, the number of remaining cells on a film base was measured to evaluate attachment properties.

According to ASTM D 3002, 5B corresponded to the case of the detachment degree of about 0%, 4B corresponded to about 5%, 3B corresponded to about 5-15%, 2B corresponded to about 15-35%, and 0B corresponded to about 35-65%.

(3) Measuring Method of Curling Property

The compensation film was cut to a size of 20 cm×20 cm (length×width) and put on a plate, and the heights from the plate to four curled sides of the film were measured. Average value was obtained (unit: mm).

(4) Measuring Antistatic Property

The sheet resistance (Ω/sq) was measured using a surface resistance measuring apparatus (Trustat Worksurface tester, ST-3) at a constant temperature and a constant humidity of 25° C. and 50%.

(5) Measuring Experiment of Resistance to High Temperature and High Humidity

After standing the compensation film in a chamber with a constant temperature and a constant humidity of 60° C. and a relative humidity of 75% for 96 hours, the generation or the migration was checked and measured.

TABLE 4

| | | | | | | High |
|---|---|---|---|---|---|---|
| | Color | | | | Anti- | temperature |
| | coordinate | | Curling | Adhesive | static | and high |
| division | CIE x | CIE y | property | strength | property | humidity |
| Preparation | 0.30 | 0.28 | <1 mm | 5B | 10¹² | No migration |
| Example 18 | | | | | | |
| Preparation | 0.29 | 0.31 | <1 mm | 5B | 10¹² | No migration |
| Example 19 | | | | | | |
| Preparation | 0.28 | 0.36 | <1 mm | 5B | 10¹² | No migration |
| Example 20 | | | | | | |
| Preparation | 0.37 | 0.27 | <1 mm | 5B | 10¹² | No migration |
| Example 21 | | | | | | |
| Preparation | 0.42 | 0.26 | <1 mm | 5B | 10¹² | No migration |
| Example 22 | | | | | | |
| Preparation | 0.45 | 0.25 | <1 mm | 5B | 10¹² | No migration |
| Example 23 | | | | | | |
| Comparative | 0.26 | 0.58 | <1 mm | 5B | 10¹² | No migration |
| Preparation | | | | | | |
| Example 1 | | | | | | |
| Comparative | 0.61 | 0.35 | <1 mm | 5B | 10¹² | No migration |
| Preparation | | | | | | |
| Example 2 | | | | | | |
| Comparative | 0.26 | 0.58 | <4 mm | 4B | 10¹² | No migration |
| Preparation | | | | | | |
| Example 3 | | | | | | |
| Comparative | 0.61 | 0.35 | <4 mm | 4B | 10¹² | No migration |
| Preparation | | | | | | |
| Example 4 | | | | | | |
| Comparative | 0.17 | 0.15 | <2 mm | 5B | 10¹² | No migration |
| Preparation | | | | | | |
| Example 5 | | | | | | |

Referring to the experimental results of Table 4, x coordinate was 0.20-0.50 and y coordinate was 0.15-0.40 for the compensation films of Preparation Examples 18-23, and it could be secured that all the compensation films had color coordinate in white under a blue light source. In addition, the compensation films of Preparation Examples 18-23 had good curling property, adhesion strength and antistatic property, and good resistance to high temperature and high humidity.

However, y coordinate deviated from 0.15-0.40 for the compensation film of Comparative Preparation Example 1, and x coordinate deviated from 0.20-0.50 for the compensation film of Comparative Preparation Example 2. Accordingly, the compensation of Comparative Preparation Example 1 exhibited pale green, and the compensation film of Comparative Preparation Example 2 exhibited scarlet.

In addition, for the compensation films of Comparative Preparation Examples 3 and 4, which used the bisphenol A epoxy diacrylate compound having two functional groups, the curling property was worse when compared to that of the preparation examples.

For the compensation film of Comparative Preparation Example 5, the amount used of the organic dots was too small, and the color coordinate of the blue light source itself was exhibited.

Through the examples and the experimental examples, it could be secured that the compensation films manufactured using the organic dots for a compensation film of the present invention and the composition for a compensation film had good physical properties. Such organic dots of the present invention are considered to replace the conventional quantum dots of an inorganic material, and may be used as a contrast medium. In addition, by applying the compensation film to an optical film, etc., an illumination apparatus, or a display, which has improved LCD efficiency and color reproducibility is expected to be provided.

1. A compensation film comprising organic dots of a unimolecular shape, having a photoluminescence (PL) wavelength of 500-680 nm.

2. The compensation film of claim 1, wherein the organic dots comprise at least one compound selected from a compound represented by the following Formula 1 or a compound represented by the following Formula 2: 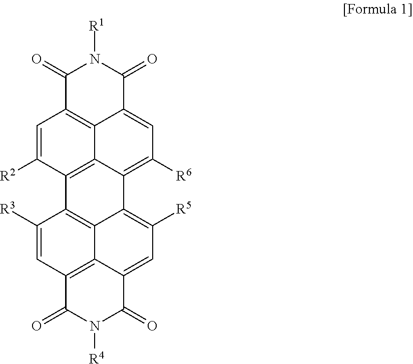
in Formula 1, R¹ and R⁴ are each independently hydrogen, linear alkyl of C1-C5, branched alkyl of C3-C5, cycloalkyl of C5-C6, 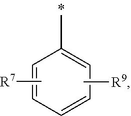
or —CN, R², R³, R⁵ and R⁶ are each independently hydrogen, alkoxy of C1-C5, cyclicalkoxy of C5-C10, 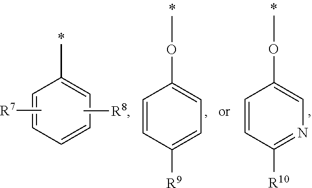
R⁷ and R⁸ are each independently hydrogen, linear alkyl of C1-C5, or branched alkyl of C3-C5, R⁹ and R¹⁰ are each independently hydrogen, —SO₃H, —COOH, —CH₂COOH, —CH₂CH₂COOH, —CH₂CH₂CH₂COOH, —NR¹¹R¹², —CH₂NR¹¹R¹², or —CH₂CH₂NR¹¹R¹², and R¹¹ and R¹² are each independently hydrogen, or linear alkyl of C1-C3, 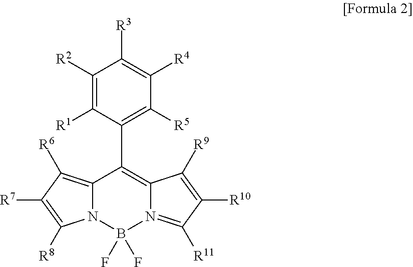
in Formula 2, R¹ to R⁵ are each independently hydrogen, alkyl of C1-C5, halogen, or —CN, R⁶ to R¹¹ are each independently hydrogen, alkyl of C1-C5, olefin of C2-C5, cycloalkyl of C5-C6, styrene, phenyl, benzyl, or —CN.

3. The compensation film of claim 2, wherein the compound represented by Formula 1 and the compound represented by Formula 2 are comprised in an amount ratio of 1:0.05-20 by weight.

4. The compensation film of claim 2, wherein in Formula 1, R¹ and R⁴ are each independently alkyl of C1-C5, or 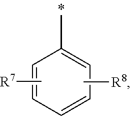
R⁷ and R⁸ are alkyl of C2-C4, or branched alkyl of C3-C4, R², R³, R⁵ and R⁶ are each independently cyclicalkoxy of C5-C10, 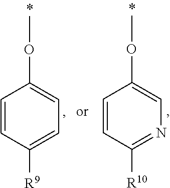
R⁹ and R¹⁰ are each independently hydrogen, —SO₃H, —COOH, —CH₂COOH, or —CH₂NR¹¹R¹², and R¹¹ and R¹² are each independently hydrogen or linear alkyl of C1.

5. The compensation film of claim 2, wherein in Formula 2, R¹ to R⁵ are each independently hydrogen, or alkyl of C1-C2, R⁷ and R¹⁰ are hydrogen, R⁶, R⁸, R⁹ and R¹¹ are each independently alkyl of C1-C2, cycloalkyl of C5-C6, styrene, phenyl, benzyl, or —CN.

6. The compensation film according to claim 1, wherein an x coordinate range is 0.20-0.50, and a y coordinate range is 0.15-0.40 on the basis of a national television system committee (NTSC) color coordinate under a blue light source.

7. The compensation film of claim 6, wherein an average thickness is 0.1-200 μm.

8. Organic dots for a compensation film, the organic dots comprising at least one compound selected from a compound represented by the following Formula 1 or a compound represented by the following Formula 2: 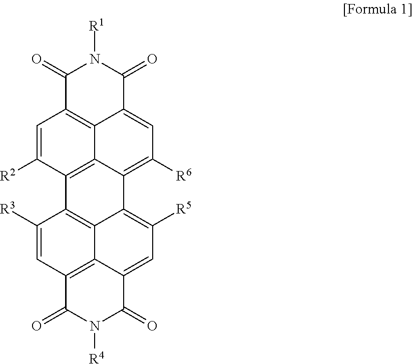
in Formula 1, R¹ and R⁴ are each independently hydrogen, linear alkyl of C1-C5, branched alkyl of C3-C5, cycloalkyl of C5-C6, 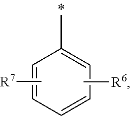
or —CN, R², R³, R⁵ and R⁶ are each independently hydrogen, alkoxy of C1-C5, cyclicalkoxy of C5-C10, 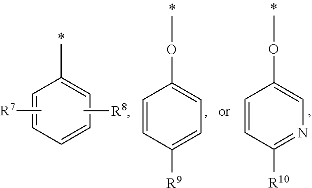
R⁷ and R⁸ are each independently hydrogen, linear alkyl of C1-C5, or branched alkyl of C3-C5, R⁹ and R¹⁰ are each independently hydrogen, —SO₃H, —COOH, —CH₂COOH, —CH₂CH₂COOH, —CH₂CH₂CH₂COOH, —NR¹¹R¹², —CH₂NR¹¹R¹², or —CH₂CH₂NR¹¹R¹², and R¹¹ and R¹² are each independently hydrogen, or linear alkyl of C1-C3, 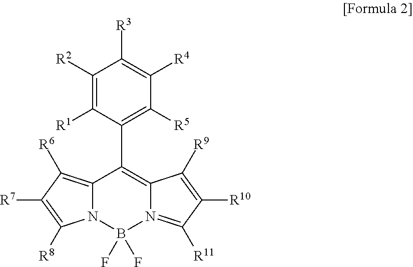
in Formula 2, R¹ to R⁵ are each independently hydrogen, alkyl of C1-C5, halogen, or —CN, R⁶ to R¹¹ are each independently hydrogen, alkyl of C1-C5, olefin of C2-C5, cycloalkyl of C5-C6, styrene, phenyl, benzyl, or —CN.

9. The organic dots for a compensation film of claim 8, wherein the compound represented by Formula 1 has a photoluminescence (PL) wavelength of 580-680 nm, and the compound represented by Formula 2 has a photoluminescence (PL) wavelength of 500-680 nm.

10. A compensation film composition, comprising 0.05-7 parts by weight of a luminescent material comprising the organic dots according to claim 8, and 30-1,700 parts by weight of beads relative to 100 parts by weight of a binder.

11. The compensation film composition of claim 10, wherein the luminescent material comprises the compound represented by Formula 1 and the compound represented by Formula 2 in a weight ratio of 1:0.05-20.

12. The compensation film composition of claim 10, wherein the binder comprises at least one selected from an aliphatic urethane acrylate resin, an epoxy acrylate resin, a melamine acrylate resin, or a polyester acrylate resin.

13. A light emitting diode (LED) display, comprising the compensation film according to claim 6.

14. A light emitting diode (LED) illumination apparatus, comprising the compensation film according to claim 6.

15. A liquid crystal display (LCD), comprising the compensation film according to claim 6.
